(19) United States
(12) Patent Application Publication
(10) Pub. No.: US 2025/0385010 A1
(43) Pub. Date: Dec. 18, 2025

(54) REMOTE HEALTH MONITORING USING PLUG-IN DEVICES WITH EVENTFUL AND STATEFUL HEALTH MONITORING

(71) Applicant: HARMAN INTERNATIONAL INDUSTRIES, INCORPORATED, Stamford, CT (US)

(72) Inventor: Anuj GUPTA, Uttar Pradesh (IN)

(21) Appl. No.: 18/878,788

(22) PCT Filed: Jun. 27, 2023
(86) PCT No.: PCT/US2023/026378

§ 371 Date: Dec. 24, 2024

(30) Foreign Application Priority Data
Jun. 28, 2022 (IN) 202241037054

(51) Int. Cl. G16H 50/30; A61B 5/00; G16H 10/60; G16H 20/10; G16H 40/67

(52) U.S. Cl. CPC G16H 50/30; A61B 5/7282; A61B 5/746; A61B 5/7465; G16H 10/60; G16H 20/10; G16H 40/67

(57) ABSTRACT

Techniques for remote healthcare monitoring using plug-in devices include receiving, from a remote source, a healthcare plan assigned to a patient; causing a separate media device to present healthcare information of the patient based on the healthcare plan; receiving healthcare data regarding the patient from one or more healthcare monitoring devices based on the healthcare plan; and sending the healthcare data to the remote source.

### CROSS-REFERENCE TO RELATED APPLICATIONS

This application claims the priority of co-pending Indian patent application titled “Remote Health Monitoring Using Plug-in Devices with Eventful and Stateful Health Monitoring,” filed on Jun. 28, 2022, and having application number 202241037054. The subject matter of this related application is hereby incorporated herein by reference.

### BACKGROUND

### Field of the Various Embodiments

Embodiments of the present invention relate generally to providing healthcare services and, more specifically, to remote health monitoring using plug-in devices with eventful and stateful health monitoring.

### Description of the Related Art

Remote health monitoring services provided via mobile applications have played a significant role in transformation of the healthcare sector. Using mobile applications, which are frequently installed on smartphones, healthcare providers (e.g., doctors and nurses) can easily connect with their patients after the patients are discharged from the hospital or leave the office and continue to provide healthcare remotely. The remote health monitoring services can involve videocalls between patients and healthcare providers for consultations and diagnoses, as well as monitoring of patient vital signs (e.g., blood pressure, pulse rate, and/or the like) via healthcare monitoring devices (e.g., wearable healthcare monitoring devices) that report patient information to the mobile applications, which provide the patient information to the remote healthcare providers via wireless or wired communication networks.

Applications on smartphones are well adapted for the delivery of remote health monitoring services because the smartphones have built-in connections to communications networks, video display screens, cameras, speakers, microphones, and the computational capacity to collect, organize, and transmit patient data. Typical smartphones also include wireless networking capabilities (e.g., Bluetooth™) that enable the smartphones to wirelessly gather patient data from healthcare monitoring devices. However, a drawback of using smartphones to monitor patients is that some patients have difficulties using smartphones. These difficulties can include an inability to use the small screens of the smartphones as input devices, due to muscular control problems that interfere with the fine motor control of the patients. These difficulties can also include an inability to read information on the screens of the smartphones, due to deteriorating vision of the patients. For these reasons, many elderly patients resist adoption of remote health monitoring applications.

Healthcare monitoring devices can measure various vital signs, monitor activity levels, and capture stressful events without user intervention by frequently collecting data on the patient at regular intervals. Such frequent measurement of vital signs generates large quantities of health data, which can be challenging for a healthcare provider and/or data analysis program to analyze, correlate, and interpret. Furthermore, with the limited battery capacity of many battery-powered healthcare monitoring devices, configuring frequent data collection leads to rapid draining of batteries, which results in frequent recharging or frequent battery replacement. To address this, healthcare monitoring devices are often configured to compromise by collecting data less often so as to reduce the quantity of data generated and improve the battery life of the healthcare monitoring devices. However, capturing data less often can cause a healthcare monitoring device to miss capturing vital data at an important moment, such as while the patient is experiencing a stressful condition or is in an important physiological state (e.g., a heart attack).

As the foregoing illustrates, what is needed in the art are systems to provide remote health monitoring services on devices with larger screens and with a capability to adaptively change the collection of data from healthcare monitoring devices with eventful and stateful health monitoring.

### SUMMARY

Various embodiments of the present disclosure set forth a computer-implemented method for remote health monitoring. The method includes receiving, from a remote source, a healthcare plan assigned to a patient; causing a separate media device to present healthcare information of the patient based on the healthcare plan; receiving healthcare data regarding the patient from one or more healthcare monitoring devices; and sending the healthcare data to the remote source.

Further embodiments provide, among other things, one or more non-transitory computer-readable media and systems configured to implement the method set forth above.

At least one technical advantage of the disclosed techniques relative to the prior art is that, with the disclosed techniques, the likelihood of a user using a remote health monitoring application is increased, which makes both the remote health monitoring application and the associated remote health monitoring platform more effective and reliable. Another advantage is that patient health data is collected at times that are relevant to the health condition of a patient while reducing power consumption, reducing memory usage, and reducing bandwidth. A further advantage is that computing resources used to analyze the collected data are reduced. These technical advantages provide one or more technological improvements over prior art approaches.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 is a schematic diagram of a remote patient monitoring (RPM) system configured to implement one or more aspects of the present disclosure;

FIG. 2 illustrates an example of a graphical user interface (GUI) for configuring a healthcare plan, according to various embodiments of the present disclosure;

FIG. 3 illustrates an example of a GUI for configuring a healthcare plan, according to various embodiments of the present disclosure;

FIG. 4 illustrates a flow diagram of method steps for remote health monitoring, according to various embodiments of the present disclosure;

FIG. 5 illustrates a flow diagram of method steps for remote health monitoring, according to various embodiments of the present disclosure; and

FIG. 6 illustrates a flow diagram of method steps for remote health monitoring, according to various embodiments of the present disclosure.

### DETAILED DESCRIPTION

In the following description, numerous specific details are set forth to provide a more thorough understanding of the various embodiments. However, it will be apparent to one of skill in the art that the inventive concepts may be practiced without one or more of these specific details.

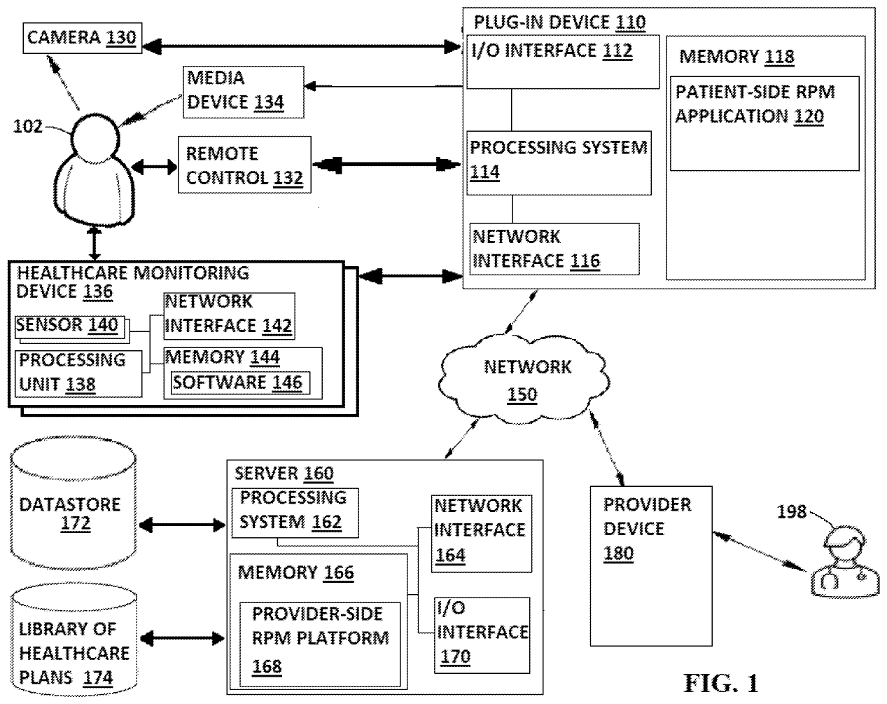

FIG. 1 is a schematic diagram of a remote patient monitoring (RPM) system 100 configured to implement one or more aspects of the present disclosure. RPM system 100 monitors and collects healthcare data regarding a patient 102 according to a healthcare plan selected and/or configured by a healthcare provider 198 (e.g., a doctor, nurse practitioner, or nurse). The RPM system 100 includes, without limitation, a plug-in device 110, one or more healthcare monitoring devices 136, a server 160, a datastore 172, a library of healthcare plans 174, and a provider device 180. The devices of the RPM system 100 communicate with each other or via a network 150 (e.g., the Internet).

The plug-in device 110 is a computer-based system that interacts with the patient 102 via various devices, including, without limitation, a media device 134, a remote control 132, a camera 130, and one or more healthcare monitoring devices 136. The plug-in device 110 includes, without limitation, an input/output (I/O) interface 112, a processing system 114, a network interface 116, and a memory 118. In some embodiments, the plug-in device 110 is an over-the-top (OTT) device, such as a Fire TV™ stick, an Apple™ TV, a Chromecast™, a Roku™, an Nvidia Shield™, and/or the like. The plug-in device 110 can include any technically feasible type of computer system.

The I/O interface 112 facilitates connectivity between the plug-in device 110 and peripheral devices. The I/O interface can include one or more of a high-definition multimedia interface (HDMI), a universal serial bus (USB) interface, an infrared (IR) interface, a radio frequency (RF) interface, a serial interface, a parallel interface, and/or the like. Examples of devices the plug-in device 110 can communicate with via the I/O interface 112 include, without limitation, a camera 130 (which can include a microphone), a media device 134 (which can include speakers), and a remote control 132 associated with the plug-in device 110.

Processing system 114 generally comprises a programmable processor that executes program instructions to manipulate input data and generate output data. Processing system 114 can be implemented as a central processing unit (CPU), a digital signal processing unit (DSP), a microprocessor, an application-specific integrated circuit (ASIC), a neural processing unit (NPU), a graphics processing unit (GPU), a field-programmable gate array (FPGA), and/or any other type of processing unit, or a combination of different processing units. The processing system 114 of the plug-in device 110 executes a patient-side remote patient monitoring application 120 that is stored in the memory 118.

Network interface 116 enables the patient-side RPM application 120 to communicate with the network 150 and via the network with a provider-side RPM platform 168 executing on a server 160. Network interface 116 also enables the patient-side RPM application 120 to communicate with one or more healthcare monitoring devices 136 (e.g., a FitBit™). Network interface 116 can implement any technically feasible wired or wireless communications protocol to facilitate the communications with the network 150 and/or the healthcare monitoring devices 136. For example, network interface 116 can include one or more of an optical network interface, a cable-network interface, an ethernet interface, a Wi-Fi transceiver, a Bluetooth™ transceiver, and/or the like.

Memory 118 includes a memory module or a collection of memory modules. Memory 118 can include a variety of computer-readable media selected for their size, relative performance, or other capabilities: volatile and/or non-volatile media, removable and/or non-removable media, and/or the like. Memory 118 can include cache, random access memory (RAM), storage, and/or the like. Memory 118 can include one or more discrete memory modules, such as dynamic RAM (DRAM) dual inline memory modules (DIMMs). In various embodiments, the non-volatile memory includes flash memory, optical drives, magnetic drives, flash drives, and/or other non-volatile storage. In some embodiments, separate data stores (not shown), can supplement memory 118.

Memory 118 generally stores application programs including patient-side RPM application 120, and data for processing by processing system 114. In some embodiments, memory 118 includes various software programs and modules (e.g., an operating system, one or more applications, and/or the like) that can be executed processing system 114 and application data associated with the software program. As shown, memory 118 includes at least the patient-side RPM application 120 that is executed by processing system 114 to implement the overall functionality of plug-in device 110 related to remote patient monitoring as discussed in greater detail herein.

Patient-side RPM application 120 configures the healthcare monitoring devices 136 and receives healthcare data captured by the healthcare monitoring devices 136. In some examples, the RPM application 120 uses an API provided by the software 146 of the healthcare monitoring device 136 to request the healthcare data from the healthcare monitoring device 136 at periodic intervals, in response to various alerts, in response to notifications received from the healthcare monitoring device 136, and/or in response to notifications received from another healthcare monitoring device 136. As illustrated, the patient-side RPM application 120 communicates with the healthcare monitoring devices 136 using the network interface 116. For example, the patient-side RPM application 120 can communicate with the healthcare monitoring devices 136 via any feasible wired (e.g., USB) or wireless (e.g., Wi-Fi or Bluetooth™) networking protocol. The patient-side RPM application 120 stores the received healthcare data in the memory 118 or alternatively in separate storage (not shown).

The patient-side RPM application 120 communicates with the provider-side RPM platform 168 via the network 150 using network interface 116. The patient-side RPM application 120 receives a healthcare plan from the provider-side RPM platform 168. In some examples, the patient-side RPM application 120 sends a confirmation of the receipt of the healthcare plan to the provider-side RPM platform 168 after receiving a healthcare plan from the provider-side RPM platform 168. The patient-side RPM application 120 sends healthcare data (e.g., healthcare data received from the healthcare monitoring devices 136 and stored in the memory 118) to the provider-side RPM platform 168.

The patient-side RPM application 120 communicates with the media device 134, the camera 130, and the remote control 132 via the I/O interface 112 and/or the network interface 116. The patient-side RPM application 120 communicates with the media device 134 to present information to the patient 102. In some examples, the information includes content related to the healthcare plan (e.g., after-visit summaries, educational material, and/or the like). The patient-side RPM application 120 presents healthcare reminders and/or healthcare alerts as needed based on the healthcare plan and patient monitoring data. The patient-side RPM application 120 communicates with the camera 130 to receive images of the patient 102, facilitate video calls with a healthcare provider, and/or the like. The patient-side RPM application 120 communicates with the remote control 132 to receive input from the patient 102, such as when used by the patient 102 to navigate and interact with a user interface presented on the media device 134 by the patient-side RPM application 120.

The server 160 is a computer-based system that supports the provider-side functionality of the RPM system 100. The server 160 includes, without limitation, a processing system 162, a memory 166, a network interface 164, and an I/O interface 170. And although server 160 is shown as a single server, it is understood that the functionality provided by server 160 can be provided by two or more servers, one or more virtual machines as part of server, as part of a cloud-based infrastructure, and/or the like.

Processing system 162 generally comprises a programmable processor that executes program instructions to manipulate input data. Processing system 162 can be implemented as a central processing unit (CPU), a digital signal processing unit (DSP), a microprocessor, an application-specific integrated circuit (ASIC), a neural processing unit (NPU), a graphics processing unit (GPU), a field-programmable gate array (FPGA), and/or any other type of processing unit, or a combination of different processing units.

Network interface 164 enables the provider-side RPM platform 168 to communicate with the patient-side RPM application 120 and one or more healthcare providers 198 using network 150. In some embodiments, the Network interface 164 also enables the provider-side RPM platform 168 to communicate with the healthcare monitoring devices 136 via network 150. Network interface 164 can implement any technically feasible wired or wireless communications protocol to facilitate the communications with the network 150 and/or the healthcare monitoring devices 136. For example, network interface 164 can include one or more of an optical network interface, a cable-network interface, an ethernet interface, a Wi-Fi transceiver, a Bluetooth™ transceiver, and/or the like.

Memory 166 includes a memory module or a collection of memory modules. Memory 166 can include a variety of computer-readable media selected for their size, relative performance, or other capabilities: volatile and/or non-volatile media, removable and/or non-removable media, and/or the like. Memory 166 can include cache, random access memory (RAM), storage, and/or the like. Memory 166 can include one or more discrete memory modules, such as dynamic RAM (DRAM) dual inline memory modules (DIMMs). In various embodiments, the non-volatile memory includes flash memory, optical drives, magnetic drives, flash drives, and/or other non-volatile storage.

Memory 166 generally stores application programs including provider-side RPM platform 168, and data for processing by processing system 162. In some embodiments, memory 166 includes various software programs and modules (e.g., an operating system, one or more applications, and/or the like) that can be executed processing system 114 and application data associated with the software program. As shown, memory 166 includes at least the provider-side RPM platform 168.

In some embodiments, separate data stores, such as datastore 172 supplement memory 166. In some examples, datastore 172 includes storage for various libraries and/or other data sources to support operation of RPM system 100. In some examples, datastore 172 is implemented on one or more storage devices (e.g., disk drives, databases, and/or the like) that are coupled to the server 160. Additionally and/or alternatively datastore 172 can be coupled to server 160 through network 150 and can include network attached storage, cloud-based storage, and/or the like.

I/O interface 170 enables the provider-side RPM platform 168 to communicate with various peripheral devices to communicate with the healthcare provider 198. Examples of devices the provider-side RPM platform 168 can communicate with via the I/O interface 170 include, without limitation, a camera (which can include a microphone), a media device (which can include speakers), a keyboard, and/or a touchscreen.

The provider-side RPM platform 168 provides one or more user interfaces that can be used by a health care provider 198 to configure a healthcare plan assigned to the patient 102. For example, the provider-side RPM platform 168 can retrieve disease-specific or diagnosis-specific healthcare plans from a library of healthcare plans 174 and present the retrieved healthcare plans to the healthcare provider 198 for the healthcare provider 198 to use in configuring and/or editing the healthcare plan assigned to the patient 102. The provider-side RPM platform 168 can also retrieve healthcare data for the patient from datastore 172 containing patient-assigned healthcare plans and patient-specific healthcare data. At the direction of the health care provider 198, when the healthcare provider 198 has completed creating or updating the healthcare plan, the provider-side RPM platform 168 transmits the health care plan to the patient-side RPM application 120 on plug-in device 110.

The provider-side RPM platform 168 further receives healthcare data for the patient 102 from the patient-side RPM application 120. The provider-side RPM platform 168 stores the received healthcare data for the patient 102 in the datastore 172. The provider-side RPM platform 168 analyzes the received healthcare data for the patient 102 to determine whether to send an alert to the healthcare provider 198 regarding an update and/or a change in health status of patient 102.

The datastore 172 stores healthcare data and one or more healthcare plans of the patient 102. The datastore 172 can include non-volatile memory, such as optical drives, magnetic drives, flash drives, and/or other non-volatile storage.

The library of healthcare plans 174 stores one or more healthcare plans that are intended to be used and can be customized for any patient 102. The library of healthcare plans 174 can include non-volatile memory, such as optical drives, magnetic drives, flash drives, or other storage. The healthcare provider 198 can refer to healthcare plans in the library of healthcare plans when creating or updating a healthcare plan assigned to the patient 102.

Each healthcare monitoring device includes, without limitation, a processing unit 138, a memory 144, a network interface 142, and one or more sensors 140. The healthcare monitoring devices 136 each receive a configuration from the patient-side RPM application 120 or from the provider-side RPM platform 168, sense the patient 102 to collect healthcare data based on the configuration, store the healthcare data, and send the healthcare data to the patient-side RPM application 120 or the provider-side RPM platform 168.

Processing unit 138 can include a central processing unit (CPU), a digital signal processing unit (DSP), a microprocessor, an application-specific integrated circuit (ASIC), a neural processing unit (NPU), a graphics processing unit (GPU), a field-programmable gate array (FPGA), and/or any other type of processor, or a combination of different processors. Processing unit 138 generally comprises a programmable processor that executes program instructions to manipulate input data. In some embodiments, processing unit 138 can include any number of processing cores, memories, and other modules for facilitating program execution.

Memory 144 includes a memory module, or collection of memory modules. Memory 144 can include a variety of computer-readable media selected for their size, relative performance, or other capabilities: volatile and/or non-volatile media, removable and/or non-removable media, and/or the like. Memory 144 can include cache, random access memory (RAM), storage, and/or the like. Memory 144 can include one or more discrete memory modules, such as dynamic RAM (DRAM) dual inline memory modules (DIMMs). In various embodiments, non-volatile memory, such as optical drives, magnetic drives, flash drives, and/or other types of non-volatile storage. In some embodiments, separate data stores can supplement memory 144.

Memory 144 generally stores application programs and data for processing by processing unit 138. In various embodiments, memory 144 stores software 146 for performing various functions or techniques described herein. The software 146 provides application program interfaces (APIs) for the healthcare monitoring device 136. The APIs can be used by the patient-side RPM application 120 and/or the provider-side RPM platform 168 to configure, access, and use the functionality provided by the healthcare monitoring device 136.

The processing unit 138 of each healthcare monitoring device 136 controls the one or more sensors 140 to collect healthcare data regarding the patient 102 based on the configuration received from the patient-side RPM application 120 or the provider-side RPM platform 168. The processing unit 138 of each healthcare monitoring device 136 also stores the healthcare data collected by the one or more sensors 140 in the memory 144. The processing unit 138 of each healthcare monitoring device 136 also sends, via the network interface 142, the stored healthcare data to the patient-side RPM application 120 or the provider-side RPM platform 168.

The one or more sensors 140 collect healthcare data of the patient 102. The one or more sensors 140 can sense, for example, the pulse rate, blood pressure, temperature, electroencephalograms (EEGs), electrocardiograms (EKGs), rapid eye movement (REM), physical activity (e.g., exercising, walking, climbing stairs), oxygenation level of the patient 102, and/or the like. The healthcare data measured by the one or more sensors is read by the processing unit 138, which stores the sensed healthcare data in the memory 144 and/or takes other actions (e.g., change a configuration of a sensor 140) in response to the sensed healthcare data.

The provider device 180 enables the healthcare provider 198 to interact with the provider-side RPM platform 168 and/or the patient-side RPM application 120. The provider device 180 interacts with the provider-side RPM platform 168 to enable the healthcare provider 198 to create, review, update, or delete a healthcare plan assigned to the patient 102. The provider device 180 also interacts with the provider-side RPM platform 168 to enable the healthcare provider 198 to review healthcare data (e.g., records of vital signs) of the patient 102 and/or to have a video call with the patient 102. The provider device 180 interacts with the patient-side RPM application 120 to review healthcare data (e.g., current vital signs) of the patient 102 and/or to have a video call with the patient 102. The provider device 180 can be a smartphone, tablet, laptop, or any other type of computing device capable of communicating via network 150.

The patient-side RPM application 120 receives a healthcare plan from the provider-side RPM platform 168 via the network 150, stores the healthcare plan in the memory 118 of the plug-in device 110, and parses the healthcare plan. The patient-side RPM application 120 parses the healthcare plan to identify and process offerings of content to the patient 102, monitoring configurations, event configurations, state configurations, reminder configurations, and/or alert configurations.

The patient-side RPM application 120 configures healthcare monitoring devices 136 according to a healthcare plan received by the patient-side remote patient monitoring application 120. Configuring a healthcare monitoring device 136 includes, without limitation, sending instructions to the healthcare monitoring device 136 to collect healthcare data regarding the patient 102 at particular times, at regular intervals, in response to detected events, or in response to detected states. In some examples, the patient-side RPM application 120 uses the one or more APIs of the healthcare monitoring device 136 to configure the healthcare monitoring device 136. Depending upon the capabilities of the healthcare monitoring device 136, the patient-side RPM application 120 can configure the healthcare monitoring device 136 to collect data from different sensors 140 either operating together or independently from each other. In some embodiments, the patient-side RPM application 120 configures a healthcare monitoring device 136 to collect a first type of healthcare data with a first sensor 140 at a first set of times and to collect a second type of healthcare data with a second sensor 140 at a second set of times. The intervals between the first set of times can be different than the intervals between the second set of times, resulting in the first sensor 140 collecting healthcare data a number of times during a period and the second sensor 140 collecting healthcare data a different number of times during the same period.

The patient-side RPM application 120 receives healthcare data from healthcare monitoring devices 136. The patient-side RPM application 120 stores the healthcare data in the memory 118 in the plug-in device 110. The patient-side RPM application 120 can additionally or alternatively forward the healthcare data to the provider-side RPM platform 168.

A healthcare plan can be assigned to the patient 102 by a healthcare provider 198 and can be customized for the patient 102 by the healthcare provider 198. A healthcare plan can include, without limitation, offerings of content to the patient 102, monitoring configurations, event configurations, state configurations, reminder configurations, and/or alert configurations.

The offerings of content in a healthcare plan includes information associated with the healthcare plan that a healthcare provider (e.g., healthcare provider 198) would like to make available to a patient (e.g., patent 102). For example, the content could include one or more of an after-visit summary, a questionnaire, a set of instructions, and/or third-party content, such as web pages, audio, video, and/or the like. The content can either be included in the healthcare plan or identified using links, such as a universal resource locator (URL). When the healthcare plan includes content to be offered to the patient, the patient-side RPM application 120 offers the content to the patient. For example, the patient-side RPM application 120 can display a user interface using media device 134 with a list of available content. The patient can use an input device (e.g., remote control 132, a microphone) to select the content to consume. The patient-side RPM application 120 then presents the content to the patient via an output device (e.g., media device 134). When the content is identified by a link or URL, the patient-side RPM application 120 uses the link or URL to retrieve the content in order to present the retrieved content to the patient. In some embodiments, if the patient 102 does not consume the content (e.g., watch an offered video or listen to an audio file) when the content is offered, the patient-side RPM application 120 can configure a reminder for the patient 102 to consume the content at a later time.

An event configuration includes, without limitation, a definition for detecting that a healthcare event has occurred a set of healthcare data to be collected and/or reported in response to detecting that the event has occurred. Examples of events that can be defined in event configurations include, without limitation, a vital sign of the patient 102 going above or below a threshold, the patient 102 falling, the patient 102 beginning or ceasing activities (e.g., exercise, climbing stairs, eating, sleeping, walking, running), and/or the like.

A state configuration includes, without limitation, a definition for detecting the patient is in a particular physiological state and a set of vital signs to be reported while the patient is in the physiological state. Examples of physiological states that can be defined in state configurations include, without limitation, being asleep, being in rapid eye movement (REM) during sleep, exercising, walking, climbing stairs, being in labor, and/or the like.

A reminder configuration causes the patient-side RPM application 120 to present a reminder to the patient 102. The patient-side RPM application 120 causes the reminder to be presented via the media device 134. The patient-side RPM application 120 can cause the reminder to be presented beside other content on the media device 134 or in a pop-up window over the other content on the media device 134. The patient-side RPM application 120 can interrupt other content on the media device 134 with the reminder and/or pause the other content while presenting the reminder. In some examples, a reminder also includes an audio component, such as a tone, a distinctive sound, a verbal component, and/or the like. A reminder includes, without limitation, a message regarding taking a medication, a message regarding an appointment with the healthcare provider 198, a message regarding an activity (e.g., sleeping, eating, or exercising) of the patient 102, a message regarding an offering of content to the patient 102, and/or the like.

An alert configuration causes the patient-side RPM application 120 to send an alert to the healthcare provider 198. The alert configuration has one or more criteria for triggering the alert and a severity of the alert (e.g., critical, high, or low). Optionally, the alert configuration can include a message to include when making the alert. Criteria for alert configurations include, without limitation, healthcare data of the patient 102 (e.g., a vital sign measured by a healthcare monitoring device 136), a lack of healthcare data of the patient 102 (e.g., a healthcare monitoring device 136 stops reporting measurements to the patient-side RPM application 120), a lack of responsiveness of the patient 102 (e.g., failure to acknowledge a reminder), and/or the like. When the alert has a severity level of “critical,” the patient-side RPM application 120 sends an immediate notification, such as a text message or automated phone call, to the healthcare provider 198. When the alert has a severity level of “high,” the RPM application 120 presents an audio and/or video alert on the provider device 180. When the alert has a severity level of “low,” the RPM application 120 causes the alert to be recorded in the datastore 172 so that the alert is presented to the healthcare provider 198 the next time the health care provider reviews the records of the patient 102.

The patient-side RPM application 120 analyzes the healthcare data received from the one or more healthcare monitoring devices 136 to detect whether an event defined by an event configuration in a healthcare plan has occurred. If the event is detected, then the patient-side RPM application 120 can configure one or more healthcare monitoring devices 136 to measure vital signs of the patient 102 according to the event configuration. If the patient-side RPM application 120 determines that one healthcare monitoring device 136 can both detect the event and measure the vital sign(s) to be reported in response to detecting the event, then the patient-side RPM application 120 configures the healthcare monitoring device 136 to detect the event and to measure the vital sign(s) in response to detecting the event. For example, an event configuration can define an event as the pulse rate of the patient being less than a threshold and blood pressure of the patient is to be measured for three minutes in response to the pulse rate falling below the threshold. In such a case, the patient-side RPM application 120 configures a healthcare monitoring device 136 to monitor the pulse rate of the patient 102 and, in response to detecting that the pulse rate is less than the threshold, measure the blood pressure for three minutes and report the measurements to the patient-side RPM application 120. In another example, an event configuration can define an event as the patient climbing three stairs and the oxygenation level of the patient is to be measured for one minute in response to the patient climbing the three stairs. In such a case, the patient-side RPM application 120 configures a healthcare monitoring device 136 to monitor the movement of the patient 102 and, in response to detecting that the patient 102 climbs three stairs, measure the oxygenation level for one minute and report the measurements to the patient-side RPM application 120.

If the patient-side RPM application 120 determines that a healthcare monitoring device 136 that is able to detect the event cannot measure the vital sign(s) to be reported in response to detecting the event, then the patient-side RPM application 120 configures a first healthcare monitoring device 136 to detect the event and collect and report healthcare data indicative of the event. If the first healthcare monitoring device 136 reports the event has occurred or the monitored healthcare data indicates that the event has occurred, then in response, the patient-side RPM application 120 configures the first healthcare monitoring device or a second healthcare monitoring device 136 to measure the vital sign(s). For example, an event configuration defines an event as the patient falling and blood pressure is to be measured for two minutes in response to the patient falling. In such a case, the patient-side RPM application 120 configures a first healthcare monitoring device 136 to detect when the patient falls or collect and report healthcare data indicative of a fall and, in response to the healthcare monitoring device 136 reporting that the patient has fallen or the reported healthcare data indicating that the patient has fallen, the patient-side RPM application 120 configures a second healthcare device 136 to collect the blood pressure of the patient for two minutes.

If the patient-side RPM application 120 determines that one healthcare monitoring device 136 can both detect the state and measure the vital sign(s) to be reported while the patient is in the state, then the patient-side RPM application 120 configures the healthcare monitoring device 136 to detect the state and to measure the vital sign(s) while the patient is in the state.

If the patient-side RPM application 120 determines that one healthcare monitoring device 136 cannot both detect the state and measure the vital sign(s) to be reported while the patient is in the state, then the patient-side RPM application 120 configures a first healthcare monitoring device 136 to detect whether the patient has entered the state or collect and report healthcare data indicative of the state. When the healthcare monitoring device 136 reports that the patient has entered the state or the monitored healthcare data indicates that the patient has entered the state, then, in response, the patient-side RPM application 120 configures the first healthcare monitoring device or a second healthcare monitoring device 136 to measure the vital sign(s) while the patient is in the state. Upon the first healthcare monitoring device 136 reporting or providing healthcare data that indicates that the patient has exited the state, the patient-side RPM application 120 reconfigures the first healthcare monitoring device or the second healthcare monitoring device 136 to stop measuring the vital sign(s), after waiting for a period (if any) included in the state configuration. In some embodiments, the patient-side RPM application 120 configures a healthcare monitoring device 136 to monitor vital sign(s) of the patient 102, and the patient-side RPM application 120 determines the patient 102 has entered the state based on the monitored vital sign(s). In some embodiments, configuring a healthcare monitoring device 136 to measure the vital sign(s) while the patient is in the state includes configuring the healthcare monitoring device 136 to collect healthcare data of the patient 102 at a higher rate than the healthcare monitoring device 136 was previously collecting healthcare data of the patient 102.

For example, a state configuration can define the state as the patient being asleep and the vital sign to be measured as the oxygenation level of the patient 102. If the patient-side RPM application 120 determines, based on data from a first healthcare monitoring device 136 that the patient 102 is in the sleep state, and, in response, the patient-side RPM application 120 configures the first healthcare monitoring device or a second healthcare monitoring device 136 to collect the oxygenation level of the patient 102 while the patient-side RPM application 120 continues to detect that the patient 102 is in the sleep state, based on data the patient-side RPM application 120 receives from the first healthcare monitoring device 136.

The patient-side RPM application 120 sends healthcare data received by the healthcare monitoring devices 136 to the provider-side RPM platform 168. The patient-side RPM application 120 sends the healthcare data based on the healthcare plan. In some examples, the healthcare plan indicates that some vital signs of patient 102 are to be checked and only reported when the vital signs are outside of a desired range, and the patient-side RPM application 120 compares the healthcare data to the desired range and determines whether to send the healthcare data to the provider-side RPM platform 168.

The patient-side RPM application 120 provides reminders to the patient 102 according to the healthcare plan assigned to the patient 102. In some examples, the reminders are presented to the patient 102 via the media device 134 and can be presented concurrently (e.g., next to or overlaying) with a television program or other content being presented by the plug-in device 110 via the media device 134.

The patient-side RPM application 120 can receive and respond to a video call initiated by the provider-side RPM platform 168. The patient-side RPM application 120 can also initiate a video call to the provider-side RPM platform 168. The patient-side RPM application 120 outputs video images and sounds of the video call via the media device 134. Similarly, the patient-side RPM application 120 accepts video images and sounds of the video call via the camera 130. The patient-side RPM application 120 sends video and audio data via the network interface 116 and network 150 to the provider-side RPM platform 168. The patient-side RPM application 120 receives video and audio data for presentation to the patient 102 via the network interface 116 and network 150.

The camera 130 collects images, video streams, and audio streams of the patient and supplies them to the patient-side RPM application 120. In some examples, the camera 130 includes a microphone. The camera 130 can be activated when the patient 102 and the healthcare provider 198 are having a video call and used to supply video and audio of the patient for the video call. During the video call, the camera 130 can be used to capture images of the patient 102 and/or one or more of the healthcare monitoring devices 136 for reference by the healthcare provider 198.

The remote control 132 communicates with the plug-in device 110 via radio signals, infrared signals, or any other technically feasible means. The remote control 132 obtains information from the patient 102 and supplies the information to the plug-in device 110. For example, the patient 102 can use the remote control 132 to scroll through a list of choices presented by the plug-in device 110 and select one of the choices. The remote control 132 includes, without limitation, push buttons and/or other means of obtaining information from the patient 102. In some embodiments, the remote control includes a microphone that can be used to capture audio commands or input from the patient.

The media device can be a television (TV), smart TV, monitor, laptop computer, tablet, or any other device capable of displaying video images and outputting audio signals. The media device 134 includes, without limitation, a video display and at least one speaker. The media device receives video and audio signals from the plug-in device 110 and converts those signals into visible and audible content.

The provider-side RPM platform 168 executes on the server 160. The provider-side RPM platform 168 enables the healthcare provider 198 to create, review, update, and/or delete a healthcare plan assigned to the patient 102. The provider-side RPM platform 168 presents one or more graphical user interfaces (GUIs), such as one or more create, review, update, and delete (CRUD) GUIs via which the healthcare provider 198 creates, reviews, updates, and/or deletes a healthcare plan assigned to the patient 102. The healthcare provider 198 can access the GUIs via the network 150 and the healthcare provider device 180. The provider-side RPM platform 168 retrieves the healthcare plan assigned to the patient 102, if any, from the datastore 172. In some examples, the provider-side RPM platform 168 also retrieves current and historic healthcare data of the patient 102 from the datastore 172 to present to the healthcare provider 198, so that the healthcare provider can refer to the healthcare data of the patient 102 when updating the healthcare plan assigned to the patient 102.

In some examples, the provider-side RPM platform 168 obtains one or more pre-configured healthcare plans, which are intended for persons having particular diseases or conditions, from a library of healthcare plans 174 and presents those one or more pre-configured healthcare plans to the healthcare provider 198. The healthcare provider 198 can then include or customize the one or more pre-configured healthcare plans to create the healthcare plan assigned to the patient 102. The provider-side RPM platform 168 selects the one or more pre-configured healthcare plans to obtain from the library of healthcare plans 174 based on input from the healthcare provider 198. When the healthcare provider 198 is satisfied with the new or updated healthcare plan to assign to the patient 102, the provider-side RPM platform 168 saves the new or updated healthcare plan assigned to the patient 102 to the datastore 172 and sends the healthcare plan to the patient-side RPM application 120.

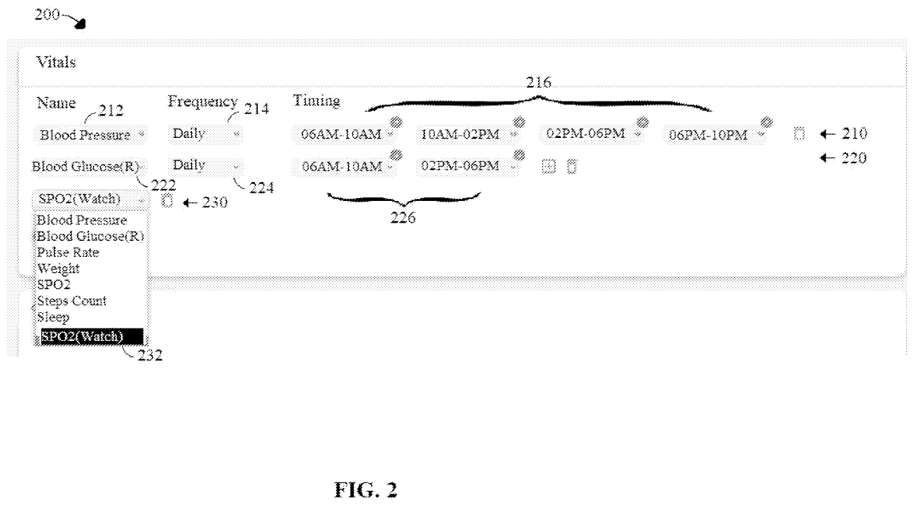

FIG. 2 illustrates an example GUI 200 for configuring a healthcare plan, according to various embodiments of the present disclosure. The GUI 200 is presented by the provider-side RPM platform 168 to allow a healthcare provider 198 to specify one or more healthcare data monitoring configurations of a healthcare plan. Each row 210, 220, or 230 in the GUI 200 illustrates a vital sign of the patient 102 that is to be monitored according to the healthcare plan. The entry at 212 shows that blood pressure is selected for monitoring in the displayed healthcare plan. The entry at 214 indicates the frequency at which the blood pressure is to be monitored. As shown via the entry at 214, the blood pressure is to be monitored daily. Although not shown, other monitoring intervals are possible, such as hourly, weekly, and/or the like. Additionally, the frequency field can indicate that the vital sign of that row is part of the criteria for an event configuration or a state configuration. When a healthcare provider 198 selects that a vital sign is part of the criteria for an event configuration, additional fields are presented by the GUI 200 to enable the healthcare provider 198 to configure vital signs to be monitored or actions to be taken in response to detecting occurrence of the event or detecting healthcare data indicative of the event. When a healthcare provider 198 selects that a vital sign is part of the criteria for a state configuration, additional fields are presented by the GUI 200 to enable the healthcare provider 198 to configure vital signs to be monitored or actions to be taken in response to detecting the physiological state or detecting healthcare data indicative of the patient entering the physiological state. The entries at 216 show that blood pressure is to be monitored between 6 AM and 10 PM. The entry at 222 shows that blood glucose (R) is selected for monitoring in the healthcare plan. The entry at 224 indicates that the blood glucose (R) is monitored daily, and the entries at 226 show that blood glucose (R) is to be monitored between 6 AM and 10 AM and between 2 PM and 6 PM. A drop-down list 232 is used by the healthcare provider 198 for selecting an additional vital sign to be monitored in the displayed healthcare plan. Once the healthcare provider 198 has selected the additional vital sign, the healthcare provider 198 selects a frequency and specific times for collecting the additional vital sign.

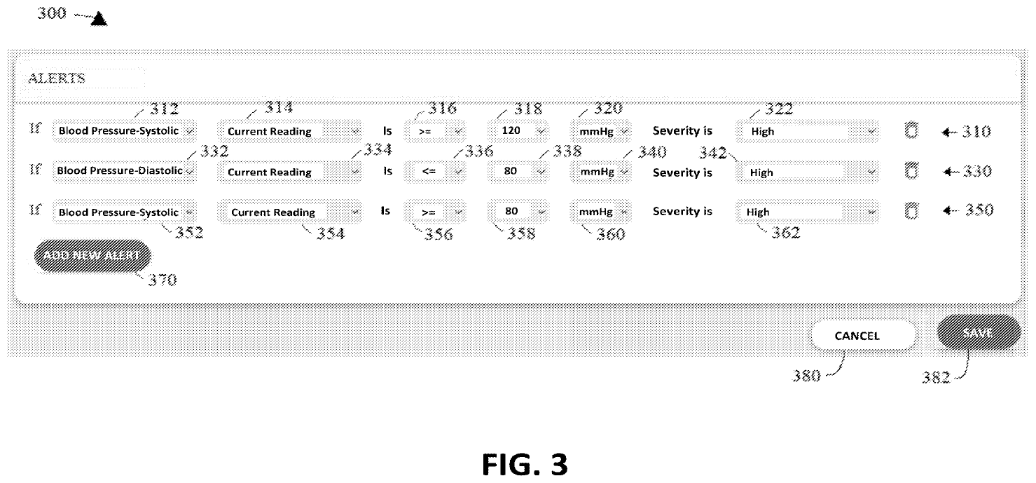

FIG. 3 illustrates an example of a GUI 300 for configuring a healthcare plan, according to various embodiments of the present disclosure. The GUI 300 is presented by the provider-side RPM platform 168 via the provider device 180 to allow a healthcare provider 198 to specify one or more alert configurations of a healthcare plan. Each row 310, 330, 350 in the GUI 300 illustrates an alert configuration included in the displayed healthcare plan.

The entry at 312 shows systolic blood pressure is selected as the basis of the first alert of the displayed healthcare plan. The entry at 314 shows which reading or function of readings on which the first alert is based. As shown via the entry at 314, the current reading of the systolic blood pressure is the basis of the first alert. Although not shown, other readings or functions of readings are possible, such as maximum reading over last hour, average reading over last two hours, or difference from a previous reading. The entry at 316 shows the relationship of the vital sign to a standard, shown at 318 and 320, for the vital sign that triggers the first alert. As shown via the entry at 316, the first alert is triggered when the current systolic blood pressure is greater than or equal to the standard shown at 318 and 320. The entry at 318 shows the numerical value for the standard for the vital sign on which the first alert is based, and the entry at 320 shows the unit of measure for the standard. As shown via the entries at 318 and 320, the standard for the first alert is 120 mmHg for the current systolic blood pressure. The entry at 322 shows the relative severity (e.g., critical, high, or low) of the first alert. The severity level of the alert determines how an RPM application, such as the RPM application 120 responds to an alert. For example, an alert with a severity level of “critical” indicates that the RPM application should provide an alert to the patient and contact emergency medical services; an alert with a severity level of “high” indicates that the RPM application should provide an alert to a patient and call or page a healthcare provider; and an alert with a severity level of “low” indicates that the RPM application should alert the patient and send a notification (e.g., an email or text message) to the healthcare provider. Each of the entries at 312, 314, 316, 318, 320, and 322 can be selected from a corresponding drop-down list.

The entry at 332 shows systolic blood pressure is selected as the basis of the second alert of the displayed healthcare plan. The entry at 334 shows which reading or function of readings on which the second alert is based. As shown via the entry at 334, the current reading of the systolic blood pressure is the basis of the second alert. The entry at 336 shows the relationship of the vital sign to a standard, shown at 338 and 340, for the vital sign that triggers the second alert. As shown via the entry at 336, the second alert is triggered when the current systolic blood pressure is less than or equal to the standard shown at 338 and 340. The entry at 338 shows the numerical value for the standard for the vital sign on which the second alert is based, and the entry at 340 shows the unit of measure for the standard. As shown via the entries at 338 and 340, the standard for the second alert is 80 mmHg for the current systolic blood pressure. The entry at 342 shows the relative severity (e.g., critical, high, or low) of the second alert. As shown via the entry at 322, the relative severity of the second alert of the displayed healthcare plan is high. Each of the entries at 332, 334, 336, 338, 340, and 342 can be selected from a corresponding drop-down list.

The entry at 352 shows systolic blood pressure is selected as the basis of the third alert of the displayed healthcare plan. The entry at 354 shows which reading or function of readings on which the third alert is based. As shown via the entry at 354, the current reading of the systolic blood pressure is the basis of the third alert. The entry at 356 shows the relationship of the vital sign to a standard, shown at 358 and 360, for the vital sign that triggers the third alert. As shown via the entry at 356, the third alert is triggered when the current systolic blood pressure is greater than or equal to the standard shown at 358 and 360. The entry at 358 shows the numerical value for the standard for the vital sign on which the third alert is based, and the entry at 360 shows the unit of measure for the standard. As shown via the entries at 358 and 360, the standard for the third alert is 80 mmHg for the current systolic blood pressure. The entry at 362 shows the relative severity (e.g., critical, high, or low) of the third alert. As shown via the entry at 322, the relative severity of the third alert of the displayed healthcare plan is high. Each of the entries at 352, 354, 356, 358, 360, and 362 can be selected from a corresponding drop-down list.

A button 370 enables the healthcare provider 198 to add a new alert to the displayed healthcare plan. Upon the healthcare provider 198 selecting the button 494, the provider-side RPM platform 168 adds a row to the GUI 300. The added row includes a set of drop-down lists for the healthcare provider 198 to use to select a vital sign, reading or function of readings, relationship to a standard, standard, unit of measure, and severity for the new alert. A cancel button 380 enables the healthcare provider 198 to exit the GUI without saving any changes to the alerts of the displayed healthcare plan. A save button 382 enables the healthcare provider 198 to save the changes to the displayed healthcare plan.

Referring again to FIG. 1, the provider-side RPM platform 168 sends the new or updated healthcare plan assigned to the patient to the patient-side RPM application 120 via the network 150 and the network interface 164. The provider-side RPM platform 168 receives a confirmation that the patient-side RPM application 120 received the new or updated healthcare plan from the patient-side RPM application 120. If the provider-side RPM platform 168 does not receive the confirmation from the patient-side RPM application 120, the provider-side RPM platform 168 can notify the healthcare provider 198 of that the patient-side RPM application 120 did not receive the new or updated healthcare plan. Optionally, the provider-side RPM platform 168 can send the healthcare plan assigned to the patient 102 to the patient-side RPM application 120 at a regular interval as a precaution against unauthorized changes at the plug-in device 110.

The provider-side RPM platform 168 receives healthcare data regarding the patient 102 from the patient-side RPM application 120. The provider-side RPM platform 168 receives the healthcare data via the network 150 and network interface 164. The provider-side RPM platform 168 saves the received healthcare data to the datastore 172.

The provider-side RPM platform 168 analyzes the healthcare data regarding the patient 102 and detects whether any alerts regarding the healthcare data regarding the patient 102 should be triggered. If the provider-side RPM platform 168 detects that an alert regarding the healthcare data regarding the patient 102 should be triggered, the provider-side RPM platform 168 sends the triggered alert to the healthcare provider 198 via a text message, email, automated phone call, or any technically feasible means.

The provider-side RPM platform 168 can initiate a video call to the patient 102, based on input from the healthcare provider 198. The provider-side RPM platform 168 can also receive a video call from the patient-side RPM application 120. The provider-side RPM platform 168 transmits a request to start a video call to the patient-side RPM application 120 via the network interface 164 and the network 150. The provider-side RPM platform 168 accepts video and audio data from the provider device 180.

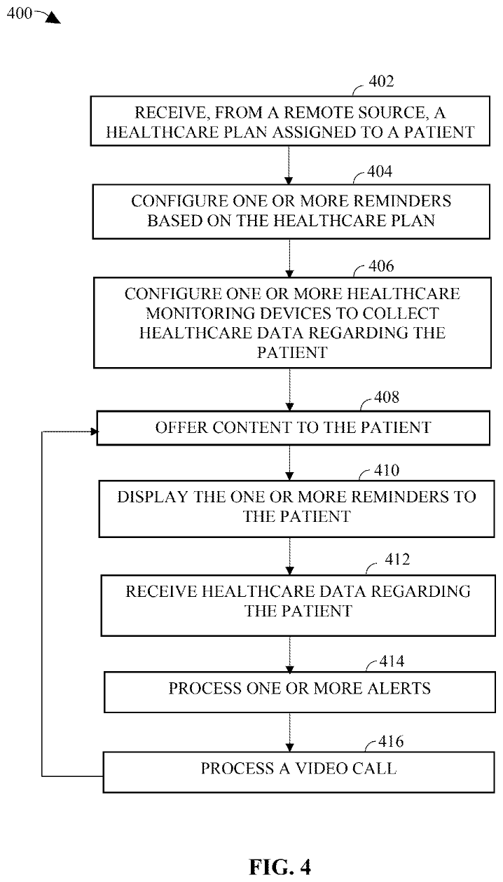

FIG. 4 illustrates a flow diagram of method steps for remote health monitoring, according to various embodiments of the present disclosure. Although the method steps are described with respect to the systems of FIG. 1, any system configured to perform the method steps, in any order, falls within the scope of the various embodiments. In some embodiments, method 400 is performed by the patient-side RPM application 120.

The method 400 begins at step 402, where a healthcare plan for a patient is received from a remote source. The health care plan includes one or more offerings of content, monitoring configurations, event configurations, state configurations, reminder configurations, and/or alert configurations, such as is described in greater detail above with respect to FIG. 1. In some examples, the healthcare plan is received by an RPM application executing on a plug-in device, such as the patient-side RPM application 120 executing on plug-in device 110. In some examples, the RPM application acknowledges receipt of the health care plan. The RPM application parses the healthcare plan to determine whether the healthcare plan includes offerings of content; configurations for healthcare monitoring devices based on monitoring configurations, event configurations, and state configurations; reminder configurations, and/or alert configurations.

In step 404, one or more reminders are configured based on the healthcare plan. The one or more reminders are configured by the RPM application. In some examples, each of the one or more reminders are a reminder to take medications or to perform some other activity at a particular time.

In step 406, one or more healthcare monitoring devices are configured to collect healthcare data regarding the patient. The one or more healthcare monitoring devices are configured by an RPM application, such as the patient-side RPM application 120. In some examples, the RPM application configures each of the one or more healthcare monitoring devices using one or more APIs provided by the healthcare monitoring device. In some examples, the one or more healthcare monitoring devices are configured per a monitoring configuration in the healthcare plan, such as to collect healthcare data for one or more vital signs at particular frequencies, collection times, and/or the like. Examples of healthcare data that can be collected include pulse rate, blood pressure, blood oxygenation, EKG, EEG, temperature, REM, physical activity (e.g., exercising, walking, climbing stairs), and/or the like. In some examples, the healthcare monitoring device is configured to detect an event or collect and report healthcare data indicative of an event per an event configuration in the healthcare plan. Examples of events include a vital sign of the patient going above or below a threshold, the patient falling, the patient beginning or ceasing activities (e.g., exercise, climbing stairs, eating, sleeping, walking, running), and/or the like. In some examples, the healthcare monitoring device is configured to detect a state and/or collect and report healthcare data indicative of a physiological state per a state configuration in the healthcare plan. Examples of physiological states include being asleep, being in rapid eye movement (REM) during sleep, exercising, walking, climbing stairs, being in labor, and/or the like. In some examples, an RPM application executing on a plug-in device configures the healthcare monitoring device via a network interface.

In step 408, content is offered to the patient. The content is offered by the RPM application via a separate media device, such as the media device 134. In some examples, offering the content includes displaying a user interface with a list of available content from which the patient can select. In some examples, the content is identified by a link or URL, and the link or URL is used by an RPM application to retrieve the content in order to present the retrieved content to the patient.

In step 410, the one or more reminders are displayed to the patient. The RPM application displays the one or more reminders via the separate media device, such as the media device 134. In some examples, each of the one or more reminders is displayed in a pop-up or alongside of other content on a separate media device, when the current time matches the time for the reminder or when other criteria for displaying the reminder are met. In some examples, each of the one or more reminders also includes an audio component, such as a tone, a distinctive sound, a verbal component, and/or the like.

In step 412, healthcare data is received regarding the patient. The RPM application receives the healthcare data regarding the patient, such as patient 102, from a healthcare monitoring device, such as healthcare monitoring device 136. In some examples, the healthcare data are received when a healthcare monitoring device detects an event or a state and begins collecting the healthcare data in response. In some examples, the healthcare data are received when a healthcare monitoring device is configured by an RPM application to start collecting healthcare data in response to the RPM application detecting an event or a state. In some examples, the RPM application analyzes the healthcare data from a first healthcare monitoring device to see whether the healthcare data indicates a particular event in an event configuration or an event in an event configuration for which the first healthcare monitoring devices is not configured to automatically collect the additional healthcare data associated with the event configuration or the state configuration. When the first healthcare monitoring device is not configured to automatically collect the additional healthcare data, the RPM application configures the first healthcare monitoring device or a second healthcare monitoring device to collect the additional healthcare data. In some examples, the RPM application uses an API provided by the healthcare monitoring device to request the healthcare data from the healthcare monitoring device at periodic intervals, in response to various alerts, in response to notifications received from the healthcare monitoring device and/or in response to notifications received from another healthcare monitoring device.

In step 414, one or more alerts are processed. In some examples, an RPM application, such as the patient-side RPM application 120, analyzes the healthcare data received during step 412 to determine whether one or more alerts are to be triggered. The RPM application compares the healthcare data to the condition identified in each alert configuration included in the healthcare plan. When the healthcare data is consistent with the condition specified in a particular alert configuration, the RPM application generates an alert consistent with the severity level specified in the particular alert configuration. Depending on the severity level, the alert includes one or more providing a notice to the patient, sending a notification to an RPM platform or a healthcare provider, dialing 911, and/or the like.

In step 416, a video call is processed. The RPM application receives a video call request from the provider-side RPM platform and/or the patient via an input, such as by the patient selecting a video call option from a GUI using a remote control. The RPM application communicates with the provider-side RPM platform 168 to set up the video call and activates the camera 130 to capture video and/or audio data from the patient 102. The RPM application sends video and audio data from the camera 130 to the provider-side RPM platform 168, receives video and audio data from the provider-side RPM platform 168, and causes the media device 134 to output the video and audio data received from the provider-side RPM platform 168.

Steps 408-416 are then repeated as needed to offer content to the patient, display one or more additional reminders, receive additional healthcare data, process one or more additional alerts, and/or support additional video calls. In some embodiments, the method 400 alternatively returns to step 402 when a new or updated healthcare plan is received.

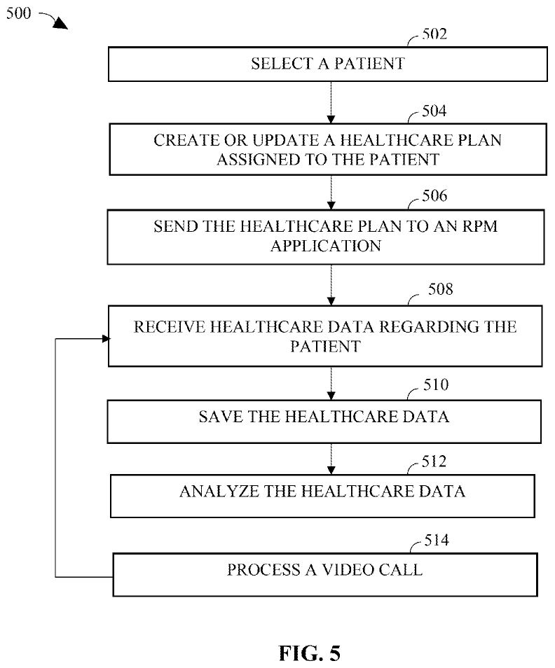

FIG. 5 illustrates a flow diagram of method steps for remote health monitoring, according to various embodiments of the present disclosure. Although the method steps are described with respect to the systems of FIG. 1, any system configured to perform the method steps, in any order, falls within the scope of the various embodiments. In some embodiments, method 500 is performed by the provider-side platform 168.

The method 500 begins at step 502, where a patient is selected. The patient is selected using an RPM platform, such as the provider-side RPM platform 168 executing on server 160. In some examples, a healthcare provider, such as healthcare provider 198, selects the patient by providing input to the RPM platform. For example, the healthcare provider can select the patient from a list of patients displayed by the RPM platform.

In step 504, a healthcare plan assigned to the patient is created or updated. The health care plan includes one or more offerings of content, monitoring configurations, event configurations, state configurations, reminder configurations, and/or alert configurations, such as is described in greater detail above with respect to FIG. 1. In some examples, a healthcare provider uses the RPM platform to create or update the healthcare plan assigned to a patient, such as the patient 102. In some embodiments, creating or updating the healthcare plan includes adding or updating one or more offerings of content, monitoring configurations, event configurations, state configurations, reminder configurations, and/or alert configurations to a pre-defined health care plan and/or a previously created health care plan of the patient. In some examples, the pre-defined healthcare plan or the previously created healthcare plan are loaded from a datastore (e.g., datastore 172) and/or a library of healthcare plans (e.g., library of healthcare plans 174). In some examples, the healthcare provider uses one or more GUIs, such as GUIs of FIGS. 2 and 3, presented by the RPM platform to create and update the healthcare plan. In some examples, the healthcare plan is stored in a datastore, such as datastore 172.

In step 506, the healthcare plan is sent to an RPM application. The RPM platform sends the healthcare plan to an RPM application, such as the patient-side RPM application 120. In some examples, the healthcare plan is sent to an RPM application in response to instructions from the healthcare provider when the healthcare provider has completed creating or updating the healthcare plan, such as the healthcare provider 198. In some examples, the healthcare plan is sent to the RPM application via a network interface, such as the network interface 164, and a network, such as the network 150. In some examples, the RPM receives a confirmation from the RPM application that the healthcare plan has been received.

In step 508, healthcare data is received regarding the patient. The RPM platform receives the healthcare data from the RPM application and/or directly from one or more healthcare monitoring devices. In some examples, the healthcare data is received in response to the RPM platform requesting the healthcare data and/or at periodic intervals.

In step 510, the healthcare data is saved. The healthcare data is saved by the RPM platform to a datastore, such as the datastore 172.

In step 512, the healthcare data is analyzed. The RPM platform analyzes the healthcare data during step 512 to determine whether one or more alerts are to be triggered. The RPM platform compares the healthcare data to the condition identified in each alert configuration included in the healthcare plan. When the healthcare data is consistent with the condition specified in a particular alert configuration, the RPM platform generates an alert consistent with the severity level specified in the particular alert configuration. Depending on the severity level, the alert includes sending a notification to a healthcare provider, dialing 911, and/or the like. In some embodiments, an alert is sent to the healthcare provider using one or more of a text message, an email, a page, a phone call, and/or the like.

In step 514, a video call is processed. The RPM platform receives a video call request from the patient-side RPM application, and/or from the healthcare provider via an input, such as by the healthcare provider selecting a video call option from a GUI using a provider device. The RPM platform communicates with the patient-side RPM application to set up the video call and activates the provider device to capture video and/or audio data from the healthcare provider. The RPM platform sends video and audio data from the provider device to the patient-side RPM application, receives video and audio data from the patient-side RPM application, and sends the video and audio data received from the patient-side RPM application to the provider device.

Steps 508-514 are then repeated as needed to receive additional healthcare data, save additional healthcare data, analyze the healthcare data, and/or support additional video calls. In some embodiments, the method 500 alternatively returns to step 502 when a new patient is selected.

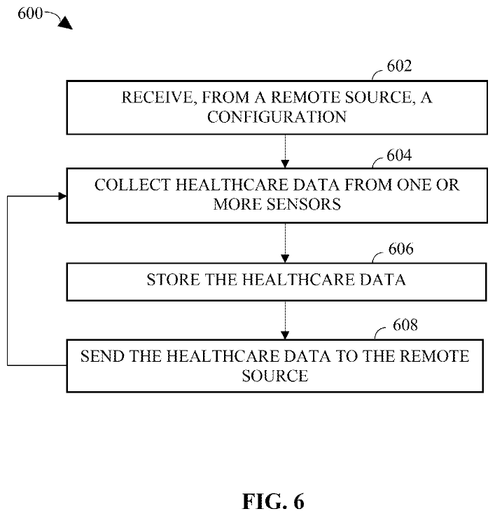

FIG. 6 illustrates a flow diagram of method steps for remote health monitoring, according to various embodiments of the present disclosure. Although the method steps are described with respect to the systems of FIG. 1, any system configured to perform the method steps, in any order, falls within the scope of the various embodiments. In some embodiments, method 600 is consistent with the operations performed by each of the healthcare monitoring devices.

The method 600 begins at step 602, where a configuration is received from a remote source. The configuration is received by a healthcare monitoring device, such as any of the healthcare monitoring devices 136. In some examples, the configuration is received from an RPM application, such as the patient-side RPM application 120, and/or from an RPM platform, such as the provider-side RPM platform 168. In some examples, the configuration is received using one or more APIs provided by the healthcare monitoring device. In some examples, the configuration is a monitoring configuration in the healthcare plan, such as to collect healthcare data for one or more vital signs at particular frequencies, collection times, and/or the like. Examples of healthcare data that can be configured to be monitored include pulse rate, blood pressure, blood oxygenation, EKG, EEG, temperature, REM, physical activity (e.g., exercising, walking, climbing stairs), and/or the like. In some examples, the configuration configures the healthcare monitoring device to detect an event or collect and report healthcare data indicative of an event per an event configuration in the healthcare plan. Examples of events include a vital sign of the patient going above or below a threshold, the patient falling, the patient beginning or ceasing activities (e.g., exercise, climbing stairs, eating, sleeping, walking, running), and/or the like. In some examples, the configuration configures the healthcare monitoring device to detect a state and/or collect and report healthcare data indicative of a physiological state per a state configuration in the healthcare plan. Examples of physiological states include being asleep, being in rapid eye movement (REM) during sleep, exercising, walking, climbing stairs, being in labor, and/or the like. In some examples, the configuration is received via a network interface.

In step 604, healthcare data is collected from one or more sensors. The healthcare data is collected by a processing unit of a healthcare monitoring device according to the configuration received during step 602. In some embodiments, the healthcare data is collected at a particular time indicated in the configuration. In some embodiments, the healthcare data is collected in response to detecting an event according to an event configuration in the configuration. In some embodiments, the healthcare data is collected in response to and while a patient is in a state according to a state configuration in the configuration.

In step 606, the healthcare data is stored. The healthcare monitoring devices stores the healthcare data in a memory and/or other storage device.

In step 608, the healthcare data is sent to the remote source. The healthcare monitoring device sends the healthcare data to the remote source via a network connection with the remote source, such as via an optical network, an ethernet LAN, a Wi-Fi LAN, a Bluetooth™ connection, and/or the like. The healthcare monitoring devices send the healthcare data to the remote source as the healthcare data is collected, at periodic intervals, when an event or state is detected, and/or in response to a request from the remote source.

Steps 604-608 are then repeated as needed to collect additional healthcare data, store the additional healthcare data, and/or send the additional healthcare data to the remote source. In some embodiments, the method 600 alternatively returns to step 602 when a new configuration is received.

In sum, a remote patient monitoring (RPM) application on a plug-in device receives, from a remote source, a healthcare plan assigned to a patient. The patient-side RPM application causes a separate media device (e.g., a television) to present healthcare information of the patient. The patient-side RPM application receives healthcare data regarding the patient from one or more healthcare monitoring devices and sends the healthcare data to the remote source. The healthcare plan includes information such as content to be provided to the patient, one or more reminders, one or more alerts, configurations for one or more healthcare monitoring devices to collect healthcare data, events to monitor for, states to be detected, and healthcare data to be collected in response to the one or more events and/or the one or more states.

At least one technical advantage of the disclosed techniques relative to the prior art is that, with the disclosed techniques, the likelihood of a user using a remote health monitoring application is increased, which makes both the remote health monitoring application and the associated remote health monitoring platform more effective and reliable. Another advantage is that patient health data is collected at times that are relevant to the health condition of a patient while reducing power consumption, reducing memory usage, and reducing bandwidth. A further advantage is that computing resources used to analyze the collected data are reduced. These technical advantages provide one or more technological improvements over prior art approaches.

1. In various embodiments, a computer-implemented method for remote patient monitoring using a plug-in device, comprises receiving, from a remote source, a healthcare plan assigned to a patient, causing a separate media device to present healthcare information of the patient based on the healthcare plan, receiving healthcare data regarding the patient from one or more healthcare monitoring devices based on the healthcare plan, and sending the healthcare data to the remote source.

2. The method of clause 1, further comprising configuring the one or more healthcare monitoring devices based on the healthcare plan.

3. The method of clause 2, wherein configuring the one or more healthcare monitoring devices comprises configuring a first healthcare monitoring device to capture a first vital sign at a first time.

4. The method of any of clauses 1-3, wherein configuring the one or more healthcare monitoring devices comprises configuring a first healthcare monitoring device to detect a physiological state of the patient and measure one or more vital signs of the patient in response to detecting the physiological state.

5. The method of any of clauses 1-4, further comprising analyzing the healthcare data to determine that the patient is in a physiological state and configuring, in response to determining that the patient is in the physiological state, a first healthcare monitoring device to capture one or more vital signs of the patient.

6. The method of any of clauses 1-5, wherein configuring the first healthcare monitoring device to capture the one or more vital signs comprises configuring the first healthcare monitoring device to capture the one or more vital signs at a first rate that is higher than a second used to capture the one or more vital signs prior to determining that the patient is in the physiological state.

7. The method of any of clauses 1-6, further comprising receiving additional healthcare data regarding the patient from the one or more healthcare monitoring devices, determining, based on the additional healthcare data, that the patient is no longer in the physiological state, and reconfiguring, in response to determining that the patient is no longer in the physiological state, the first healthcare monitoring device to stop capturing the one or more vital signs of the patient.

8. The method of any of clauses 1-7, wherein configuring the one or more healthcare monitoring devices comprises configuring a first healthcare monitoring device to detect an event associated with the patient and measure one or more vital signs of the patient in response to detecting the event.

9. The method of any of clauses 1-8, further comprising detecting occurrence of an event associated with the patient, based on the healthcare data and configuring, in response to detecting the event, a first healthcare monitoring device to capture one or more vital signs of the patient.

10. The method of any of clauses 1-9, further comprising determining, based on the healthcare data, that one or more criteria of an alert are satisfied and generating the alert in response to determining that the one or more criteria of the alert are satisfied.

11. The method of any of clauses 1-10, further comprising providing a reminder to the patient using the media device based on the healthcare plan.

12. In various embodiments, one or more non-transitory computer-readable media storing instructions that, when executed by one or more processors, cause the one or more processors to perform the steps of receiving, from a remote source, a healthcare plan assigned to a patient, causing a separate media device to present healthcare information of the patient based on the healthcare plan, receiving healthcare data regarding the patient from one or more healthcare monitoring devices based on the healthcare plan, and sending the healthcare data to the remote source.

13. The one or more non-transitory computer-readable media of clause 12, wherein the steps further comprise configuring the one or more healthcare monitoring devices based on the healthcare plan, wherein configuring the one or more healthcare monitoring devices comprises one or more of configuring a first healthcare monitoring device to capture a first vital sign at a first time, configuring the first healthcare monitoring device to measure one or more vital signs of the patient in response to detecting a physiological state of the patient, or configuring the first healthcare monitoring device to measure one or more vital signs of the patient in response to detecting an event associated with the patient.

14. The one or more non-transitory computer-readable media of clause 12 or clause 13, wherein the steps further comprise providing a reminder to the patient using the media device based on the healthcare plan.

15. The one or more non-transitory computer-readable media of any of clauses 12-14, wherein the reminder is a reminder to take a medication or to perform an action.

16. The one or more non-transitory computer-readable media of any of clauses 12-15, wherein providing the reminder comprises pausing media content being presented by the media device.

17. The one or more non-transitory computer-readable media of any of clauses 12-16, wherein the steps further comprise receiving, from a remote control associated with the plug-in device, an acknowledgment of the reminder, and restarting presentation of the content in response to receiving the acknowledgment.

18. The one or more non-transitory computer-readable media of any of clauses 12-17, wherein the steps further comprise facilitating a video call with a healthcare provider.

19. The one or more non-transitory computer-readable media of any of clauses 12-18, wherein the remote source is a remote patient monitoring platform.

20. In various embodiments, a plug-in device comprising a memory storing instructions, and a processing system coupled to the memory that executes the instructions to perform steps comprising receiving, from a remote source, a healthcare plan assigned to a patient, causing a separate media device to present healthcare information of the patient based on the healthcare plan, receiving healthcare data regarding the patient from one or more healthcare monitoring devices based on the healthcare plan, and sending the healthcare data to the remote source.

Any and all combinations of any of the claim elements recited in any of the claims and/or any elements described in this application, in any fashion, fall within the contemplated scope of the present invention and protection.

The descriptions of the various embodiments have been presented for purposes of illustration but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method, or computer program product. Accordingly, aspects of the present disclosure may take the form of an entirely hardware embodiment, an entirely software embodiment (including firmware, resident software, micro-code, and/or the like) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A computer-implemented method for remote patient monitoring using a plug-in device, comprising:
receiving, from a remote source, a healthcare plan assigned to a patient;
causing a separate media device to present healthcare information of the patient based on the healthcare plan;
receiving healthcare data regarding the patient from one or more healthcare monitoring devices based on the healthcare plan; and
sending the healthcare data to the remote source.

2. The method of claim 1, further comprising configuring the one or more healthcare monitoring devices based on the healthcare plan.

3. The method of claim 2, wherein configuring the one or more healthcare monitoring devices comprises configuring a first healthcare monitoring device to capture a first vital sign at a first time.

4. The method of claim 1, wherein configuring the one or more healthcare monitoring devices comprises configuring a first healthcare monitoring device to:
detect a physiological state of the patient; and
measure one or more vital signs of the patient in response to detecting the physiological state.

5. The method of claim 1, further comprising:
analyzing the healthcare data to determine that the patient is in a physiological state; and
configuring, in response to determining that the patient is in the physiological state, a first healthcare monitoring device to capture one or more vital signs of the patient.

6. The method of claim 5, wherein configuring the first healthcare monitoring device to capture the one or more vital signs comprises configuring the first healthcare monitoring device to capture the one or more vital signs at a first rate that is higher than a second used to capture the one or more vital signs prior to determining that the patient is in the physiological state.

7. The method of claim 5, further comprising:
receiving additional healthcare data regarding the patient from the one or more healthcare monitoring devices;
determining, based on the additional healthcare data, that the patient is no longer in the physiological state; and
reconfiguring, in response to determining that the patient is no longer in the physiological state, the first healthcare monitoring device to stop capturing the one or more vital signs of the patient.

8. The method of claim 1, wherein configuring the one or more healthcare monitoring devices comprises configuring a first healthcare monitoring device to:
detect an event associated with the patient; and
measure one or more vital signs of the patient in response to detecting the event.

9. The method of claim 1, further comprising:
detecting occurrence of an event associated with the patient, based on the healthcare data; and
configuring, in response to detecting the event, a first healthcare monitoring device to capture one or more vital signs of the patient.

10. The method of claim 1, further comprising:
determining, based on the healthcare data, that one or more criteria of an alert are satisfied; and
generating the alert in response to determining that the one or more criteria of the alert are satisfied.

11. The method of claim 1, further comprising providing a reminder to the patient using the media device based on the healthcare plan.

12. One or more non-transitory computer-readable media storing instructions that, when executed by one or more processors, cause the one or more processors to perform the steps of:
receiving, from a remote source, a healthcare plan assigned to a patient;
causing a separate media device to present healthcare information of the patient based on the healthcare plan;
receiving healthcare data regarding the patient from one or more healthcare monitoring devices based on the healthcare plan; and
sending the healthcare data to the remote source.

13. The one or more non-transitory computer-readable media of claim 12, wherein the steps further comprise configuring the one or more healthcare monitoring devices based on the healthcare plan, wherein configuring the one or more healthcare monitoring devices comprises one or more of:
configuring a first healthcare monitoring device to capture a first vital sign at a first time;
configuring the first healthcare monitoring device to measure one or more vital signs of the patient in response to detecting a physiological state of the patient; or
configuring the first healthcare monitoring device to measure one or more vital signs of the patient in response to detecting an event associated with the patient.

14. The one or more non-transitory computer-readable media of claim 12, wherein the steps further comprise providing a reminder to the patient using the media device based on the healthcare plan.

15. The one or more non-transitory computer-readable media of claim 14, wherein the reminder is a reminder to take a medication or to perform an action.

16. The one or more non-transitory computer-readable media of claim 14, wherein providing the reminder comprises pausing media content being presented by the media device.

17. The one or more non-transitory computer-readable media of claim 14, wherein the steps further comprise:
receiving, from a remote control associated with the plug-in device, an acknowledgment of the reminder; and
restarting presentation of the content in response to receiving the acknowledgment.

18. The one or more non-transitory computer-readable media of claim 12, wherein the steps further comprise facilitating a video call with a healthcare provider.

19. The one or more non-transitory computer-readable media of claim 12, wherein the remote source is a remote patient monitoring platform.

20. A plug-in device comprising:
a memory storing instructions; and
a processing system coupled to the memory that executes the instructions to perform steps comprising:
receiving, from a remote source, a healthcare plan assigned to a patient;
causing a separate media device to present healthcare information of the patient based on the healthcare plan;
receiving healthcare data regarding the patient from one or more healthcare monitoring devices based on the healthcare plan; and
sending the healthcare data to the remote source.
